SEQUENCE LISTING (1 sequence)

SEQ ID NO: 1 - DNA, 2403 bp, Artificial sequence
  Plasmid pMHas5
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaattcacagctatgctagagcggccgctcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcg

(19) United States
(12) Patent Application Publication
(10) Pub. No.: US 2006/0014149 A1
(43) Pub. Date: Jan. 19, 2006

(54) Methods for rolling circle amplification and signal trapping of libraries

(75) Inventor: Kirk Schnorr, Holte (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/523,682

(22) PCT Filed: Aug. 1, 2003
(86) PCT No.: PCT/DK03/00519

§ 371 Date: Feb. 1, 2005

(30) Foreign Application Priority Data
Aug. 1, 2002 (DK) PA 2002 01163

(51) Int. Cl. C12Q 1/68; C12N 15/74; C12N 15/85

(52) U.S. Cl. 435/6; 435/455; 435/483; 435/471

(57) ABSTRACT

A method for isolating a polynucleotide that encodes a polypeptide of interest which comprises a signal sequence for secretion or partial secretion, utilizing rolling circle amplification and signal trapping of libraries.

### FIELD OF INVENTION

A method for isolating genes encoding secreted polypeptides from rolling circle amplified gene libraries is described, in which the endogenous secretion signal sequences are detected using an in vitro transposition reaction, where the transposon comprises a secretion reporter.

### BACKGROUND OF THE INVENTION

Secreted polypeptides are highly interesting for the biotechnological and pharmaceutical industries, since they can be produced recombinantly with a minimum of purification steps necessary. A positive screening system which selects only clones encoding secreted polypeptides is thus very desirable. Signal trapping is used to identify genes encoding polypeptides that comprise a signal peptide by applying a translational fusion to an extracellular reporter encoding gene lacking its own signal. Methods and protocols for using signal trapping were disclosed in WO 01/77315 (Novozymes A/S).

Cloning and gene library construction using rolling circle amplification, mainly for sequencing purposes of in particular non-clonable targets, was disclosed by P. M. Lizardi (U.S. Pat. No. 6,287,824; U.S. Pat. No. 6,280,949), and by Dean et al. (2001, Rapid Amplification of plasmid and phage DNA using Phi29 DNA polymerase and multiply-primed rolling circle amplification. Genome Research 11:1095-1099). Commercial kits are available comprising Phi29 DNA polymerase, for use in rolling circle amplification sequencing, e.g. TempliPhi™ DNA Sequencing Template Amplification Kit (Amersham Biosciences, USA).

The construction of gene libraries for cloning or screening traditionally employs intermediate amplification host cells, e.g. Escherichia coli, because the transformation efficiency is very low with raw ligations, especially into eukaryotic host cells. The use of intermediate host cells for amplifying and/or maintaining a gene library increases the likelihood of the library not being representative of the organism it was prepared from. There may well be a bias against genetic material, the presence of which could be inhibitory or even lethal to the intermediate host, e.g. genes encoding anti-microbial activities may be lost from the library.

### SUMMARY OF THE INVENTION

The problem to be solved by the present invention is how to identify those clones in a gene library that encode efficiently secreted or surface-displayed polypeptides, even polypeptides with unknown activity, without having to redone the library into a screening-vector, without having to amplify the library in an intermediate host, and without having to screen the library in traditional labour—and time consuming activity assays that would detect known activities only. Solving this problem allows rapid and efficient industrial exploitation of relevant s secreted or surface-displayed polypeptides from new organisms.

We describe methods for signal trapping a gene library with a signal-less reporter gene, combined with an in vitro rolling circle amplification procedure allowing a sufficiently high transformation efficiency to circumvent the need for intermediate amplification host cells, thereby enabling the efficient identification and isolation of a polynucleotide encoding a polypeptide of interest which comprises a signal sequence.

Accordingly, in a first aspect the invention relates to a method for isolating a polynucleotide that encodes a polypeptide of interest which comprises a signal sequence for secretion or partial secretion, the method comprising the sequential steps of:
a) providing a DNA or cDNA library from an organism producing the polypeptide of interest, wherein the library is comprised in a circular vector and is produced in vitro without ultraviolet irradiation of the component polynucleotides;
b) amplifying the library by rolling circle amplification, thereby forming concatamers;
c) inserting into the library a DNA fragment comprising a promoterless and secretion signal-less polynucleotide encoding a secretion reporter;
d) introducing the amplified library comprising the inserted DNA fragment into a host cell;
e) screening for and selecting a host cell that secretes or partially secretes the active secretion reporter; and
f) identifying from the selected host cell the polynucleotide into which the secretion reporter was inserted, and isolating the polynucleotide;
   wherein steps b) and c) may be performed in any order.

The terms “secretes”, “partially secretes”, or “membrane displayed” are used interchangeably herein and mean translocation of a part of a polypeptide or of a whole polypeptide across a membrane of a cell such as a prokaryotic, eukaryotic, or archaea cell. In a non-limiting example of polypeptide secretion, a membrane-bound or transmembrane protein such as a receptor may in the method of the invention be expressed in a host cell as a fusion polypeptide that is fused with the “secretion reporter” of the invention; thus “secretion” in this context means translocation of the fusion polypeptide across a membrane of the host cell to such an extent that at least the secretion reporter part of the fusion polypeptide is displayed on the extracellular side of the membrane and is functionally active in a secretion reporter assay. In other examples the fusion polypeptide may be completely secreted into the cultivation media without any residual linkage to the secreting cell.

In a non-limiting example herein, cDNA or genomic DNA libraries are tagged with a transposon containing a reporter gene. All in-frame fusions of the transposon reporter gene with a gene in the library containing a signal sequence are detected by assaying the expression of active reporter. The upstream and downstream flanking DNA sequences of the transposon insertion are then sequenced and the gene into which the transposon was inserted is identified by sequence analysis. In many cases, obtaining the full sequence of a tagged gene will be facilitated by the recovery of numerous clones of the same gene tagged in different nucleotide positions or sites. Positive clones are sequenced to identify clones that represent the same gene but have different transposon insertion sites. In this way all or most of the open reading frame (ORF) can be obtained by contig assembly. If a complete ORF cannot be obtained in this manner, perhaps due to an insufficient number or an uneven distribution of transposon inserts in the gene, then the full length gene may be obtained by classical primer walking DNA sequencing.

The sequence information thus obtained can then be used to isolate the complete gene of interest including the sequence encoding the secretion signal sequence and further to make an optimal expression construct for industrial production of the secreted proteins, all well within the skill of the art, whereafter the industrial production process of expressing and recovering the enzyme is a matter thoroughly described in the art as shown elsewhere herein.

In a second aspect the invention relates to a polynucleotide encoding a polypeptide of interest, wherein said polynucleotide is isolated by the method of the present invention. A third aspect of the invention relates to a polypeptide of interest which is encoded by a polynucleotide as defined in the second aspect. A fourth aspect relates to an expression system comprising a polynucleotide as defined in the second aspect. In a fifth aspect the invention relates to a host cell comprising at least one copy of a polynucleotide as defined in the second aspect, or an expression system as defined in the fourth aspect. A final aspect relates to a process for producing a polypeptide of interest, comprising cultivating a host cell as defined in the previous aspect under conditions suitable for expressing the polynucleotide as defined in the second aspect, wherein said host cell secretes the polypeptide encoded by said polynucleotide into the growth medium.

### DEFINITIONS

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989“) DNA Cloning: A Practical Approach, Volumes I and II/D. N. Glover ed. 1985); Oligonucleotide Synthesis (M. J. Gait ed. 1984); Nucleic Acid Hybridization (B. D. Hames & S. J. Higgins eds (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984).

When applied to a protein, the term “isolated” indicates that the protein is found in a condition other than its native environment, such as apart from blood and animal tissue. In a preferred form, the isolated protein is substantially free of other proteins, particularly other proteins of animal origin. It is preferred to provide the proteins in a highly purified form, i.e., greater than 95% pure, more preferably greater than 99% pure. When applied to a polynucleofide molecule, the term “isolated” indicates that the molecule is removed from its natural genetic milieu, and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, and may include naturally occurring 5′ and 3′ untranslated regions such as promoters and terminators. The identification of associated regions will be evident to one of ordinary skill in the art (see for example, Dynan and Tijan, Nature 316: 774-78, 1985).

A “polynucleotide” is a single- or double-stranded polymer of deoxyribonucleotide or ribonucleotide bases, the sequence of a polynucleotide is read from the 5′ to the 3′ end. Polynucleotides include RNA and DNA, and may be isolated from natural sources, synthesized in vitro, or prepared from a combination of natural and synthetic molecules. A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary or quaternary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

### DETAILED DESCRIPTION OF THE INVENTION

The present invention allows the screening of genebanks or -libraries by proxy, for genes encoding secreted polypeptides or enzymes even of unknown activity and thus without known screening assays including polypeptides having antimicrobial or biocidal activity. The is methods of the invention enable screening for polypeptides of potential industrial interest that would not likely have been isolated using conventional screening assays.

A method for isolating a polynucleotide that encodes a polypeptide of interest which comprises a signal sequence for secretion or partial secretion, the method comprising the sequential steps of:
a) providing a DNA or cDNA library from an organism producing the polypeptide of interest, wherein the library is comprised in a circular vector and is produced in vitro without ultraviolet irradiation of the component polynucleotides;
b) amplifying the library by rolling circle amplification, thereby forming concatamers;
c) inserting into the library a DNA fragment comprising a promoterless and secretion signal-less polynucleotide encoding a secretion reporter;
d) introducing the amplified library comprising the inserted DNA fragment into a host cell;
e) screening for and selecting a host cell that secretes or partially secretes the active secretion reporter; and
f) identifying from the selected host cell the polynucleotide into which the secretion reporter was inserted, and isolating the polynucleotide;
   wherein steps b) and c) may be performed in any order.

The present invention can be performed using any gene libraries known in the art, specifically it can also be used with gene libraries of viable but non-culturable organisms as typically seen in environmental samples. Processes of producing representative or normalized gene-libraries from environmental samples containing non-culturable organisms have been described in the art (U.S. Pat. No. 5,763,239). Accordingly a preferred embodiment of the present invention relates to a method of the first aspect, wherein the cDNA or the cDNA library is normalized or wherein the DNA or the cDNA library is normalized.

A preferred embodiment relates to a method of the first aspect, wherein genomic DNA library or cDNA library is derived from a microorganism; preferably the microorganism is a fungus, a filamentous fungus or a yeast; more preferably the microorganism is a bacterium, or the microorganism is an archaeon.

Another preferred embodiment relates to a method of the first aspect, wherein the genomic DNA library or cDNA library is derived from a multicellular organism, preferably from a mammalian cell most preferably from a human cell.

Vectors

The present invention also relates to recombinant vectors comprising a nucleic acid sequence, of the present invention, a promoter, and transcriptional and translational stop signals. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present invention may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid, phagemid, cosmid, or virus) which can be conveniently subjected to recombinant DNA procedures and can bring about the expression of the nucleic acid sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which i has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vectors of the present invention preferably contain an element(s) that permits stable integration of the vector into the host cell's genome or autonomous replication of the vector in the cell independent of the genome.

For integration into the host cell genome, the vector may rely on the nucleic acid sequence encoding the polypeptide or any other element of the vector for stable integration of the vector into the genome by homologous or nonhomologous recombination. Alternatively, the vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its functioning temperature-sensitive in the host cell (see, e.g., Ehrlich, 1978, Proceedings of the National Academy of Sciences USA 75: 1433).

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Since the rolling circle amplification of the invention generates concatamers as an end-product, it may be advantageous to convert the concatamers to monomers. This monomerization may be achieved in any manner which is convenient, e.g. by introducing at least one restriction enzyme cleavage site in the vector for subsequent restriction and circularization; or by introducing at least one cos site to enable cleavage during phage packaging; or by introducing at least one recombination recognition site, such as the Flp recombinase recognition site, for subsequent looping out of monomers using the Flp enzyme. Accordingly, in a preferred embodiment the invention relates to a method of the first aspect, wherein the vector comprises at least one restriction enzyme cleavage site and/or at least one cos site and/or at least one recombination recognition site.

Another preferred embodiment of the invention relates to a method of the first aspect, wherein the amplified library concatamers are converted to monomers before performing step d); preferably the vector comprises at least one restriction enzyme recognition site, and the concatamers are converted to monomers by restriction enzyme digestion and then circularized by ligation, or preferably the vector comprises at least one recombination recognition site, and the concatamers are converted to monomers by circularization through homologous recombination, mediated by the recognition sites and a specific recombinase.

It is well known in the art, that supercoiled circular polynucleotides have a higher transformation efficiency than their relaxed counterparts. Accordingly, in a preferred embodiment the invention relates to a method of the first aspect, wherein the monomers are circularized and then treated with a DNA topoisomerase.

Yet another preferred embodiment of the invention relates to a method of the first aspect, wherein the vector comprises at least one cos site, and wherein subsequent to steps b) and c) the amplified library concatamers are converted to monomers prior to step d) by cos site cleavage during phage-packaging.

In some instances, better transformation efficiency is achieved when concatamers are used rather than monomers, e.g. for many Bacillus cells this is the case. Accordingly, one preferred embodiment of the invention relates to a method of the first aspect, wherein the library is introduced into the host cell as concatamers.

A DNA fragment comprising a promoterless and secretion signal-less polynucleotide encoding a secretion reporter is inserted into the library in step c) of the method of the first aspect. This may be done by an in vitro process, such as described in WO 01/77315.

So, a preferred embodiment of the invention relates to a method of the first aspect, wherein step c) is performed in vitro. A further preferred embodiment of the invention relates to a method of the first aspect, wherein the DNA fragment of comprises a transposon, preferably a MuA transposon. Still another preferred embodiment of the invention relates to a method of the first aspect, wherein the DNA fragment comprises an origin of replication which is functional in the host cell, preferably the origin of replication is functional in Escherichia coli, more preferably the origin of replication is a derivative of colE1, oriV, P15A, or colDF1 3, and most preferably the origin of replication is colE1.

Another preferred embodiment of the invention relates to a method of the first aspect, s wherein the secretion reporter is a protein which, when secreted from the host cell, allows said cell to grow in the presence of a substance which otherwise inhibits growth of said cell; preferably the secretion reporter is a β-lactamase or an invertase.

In still another preferred embodiment, the invention relates to a method of the first aspect, wherein the polynucleotide of the DNA-fragment of steb (b) encodes a secretion reporter carrying an N-terminal peptide linker which comprises a specific target site for proteolytic cleavage.

Host Cells

The present invention also relates to recombinant host cells, which are advantageously used in the method of the first aspects of the invention as well as in recombinant production of the polypeptides encoded by the gene of interest identified in the method of the invention. A vector comprising a nucleic acid sequence or gene of interest of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell for these purposes will to a large extent depend upon the gene encoding the polypeptide and its source.

The host cell may be a unicellular microorganism, erg., a prokaryote, or a non-unicellular microorganism, e.g., a eukaryote.

Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. in a preferred embodiment, the bacterial host cell is a Bacillus lentus, Bacillus licheniformis, Bacillus stearothermophilus, or Bacillus subtilis cell. In another preferred embodiment, the Bacillus cell is an alkalophilic Bacillus.

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823829, or Dubnau and Davidoff-Abelson, 1971, Journal of Molecular Biology 56: 209-221), electroporaton (see, e.g., Shigekawa and Dower, 1988, Biotechniques 6: 742-751), or conjugation (see, e.g., Koehler and Thorne, 1987, Journal of Bacteriology 169: 5771-5278).

The host cell may be a eukaryote, such as a mammalian, insect, plant, or fungal cell. In a preferred embodiment, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in is Hawksworth et al., 1995, supra, page 171) and all mitosporic fungi (Hawksworth et al., 1995, supra).

In a more preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9,1980).

In an even more preferred embodiment, the yeast host cell is a Candida, Hansenula, Kluyveromyces, Pichia, Saccharomyces, Schizosaccharomyces, or Yarrowia cell. In a most preferred embodiment, the yeast host cell is a Saccharomyces carisbergensis, Saccharomyces cerevisiae, Saccharomyces diastaticus, Saccharomyces douglasli, Saccharomyces kluyveri, Saccharomyces norbensis or Saccharomyces oviformis cell. In another most preferred embodiment, the yeast host cell is a Kluyveromyces lactis cell. In another most preferred embodiment, the yeast host cell is a Yarrowia lipolytica cell.

In another more preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In an even more preferred embodiment, the filamentous fungal host cell is a cell of a species s of, but not limited to, Acremonium, Aspergillus, Fusarium, Humicola, Mucor, Myceliophthora, Neurospora, Penicillium, Thielavia, Tolypocladium, or Trichoderma.

In a most preferred embodiment, the filamentous fungal host cell is an Aspergillus awamori, Aspergillus foetidus, Aspergillus japonicus, Aspergillus nidulans, Aspergillus niger or Aspergillus oryzae cell. In another most preferred embodiment, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In an even most preferred embodiment, the filamentous fungal parent cell is a Fusarium venenatum (Nirenberg sp. nov.) cell. In another most preferred embodiment, the filamentous fungal host cell is a Humicola insolens, Humicola lanuginosa, Mucor miehei, Myceliophthora thermophila, Neurospora crassa, Penicillium purpurogenum, Thielavia terrestris, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474.

Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

Accordingly, in a preferred embodiment the invention relates to a method of the first aspect, wherein the host cell is bacterial; preferably the bacterial host cell is an Escherichia, Lactococcus, Streptomyces, Enterococcus or Bacillus cell, preferably of the species Escherichia coli, Lactococcus lactis, Streptomyces griseus, Streptomyces coelicor, Enterococcus faecalis, Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, or Bacillus thuringiensis.

In another embodiment the invention relates to a method of the first aspect, wherein the host cell is fungal; preferably the fungal host cell is of the genus Candida, Kluyveromyces, Pichia, Saccharomyces, Schizosaccharomyces, Yarrowia, Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma; more preferably the fungal host cell is of the species Saccharomyces cerevisiae, Aspergillus aculeatus, Aspergillus awamori, Aspergillus nidulans, Aspergillus niger, or Aspergillus oryzae.

In yet another preferred embodiment the invention relates to a method of the first aspect, wherein the host cell is mammalian.

When a host cell has been selected according to the method of the invention, it is of interest to identify the polynucleotide into which the secretion reporter was inserted. There are many ways in the art that allow such an identification, e.g. hybridizations, probing, subcloning etc. An efficient way is to perform a polynucleotide sequencing of the DNA flanking the inserted DNA fragment, the sequence of which is already known. So, a preferred embodiment of the invention relates to a method of the first aspect, wherein the identifying of the polynucleotide in step f) is done by DNA sequencing using at least one primer directed to the DNA fragment of step c), or using at least one primer directed to the vector of step a); preferably where isolating the polynucleotide in step f) is done by utilizing the DNA sequence information obtained.

Once sequence information has been obtained on the polynucleotide encoding the polypeptide of interest, then the complete encoding gene may be isolated, either from the genome of the originating organism, or from the previously established library. Accordingly, a preferred embodiment of the invention relates to a method of the first aspect, wherein the polynucleotide in step f) is isolated from the genome of the organism producing the polypeptide of interest, or from a DNA or cDNA library of the organism.

A great number of secreted or partially secreted polypeptides are of commercial or industrial interest. A preferred embodiment of the invention relates to a method of the first aspect, wherein the polypeptide of interest is an enzyme that is secreted from the host cell.

Another preferred embodiment of the invention relates to a method of the first aspect, wherein the polypeptide of interest is a membrane-bound receptor, preferably a two-component signal (TCS) transduction receptor, and more preferably a cytokine receptor. Yet another preferred embodiment of the invention relates to a method of the first aspect, wherein the polypeptide of interest is a secreted cytokine. Still another preferred embodiment of the invention relates to a method of the first aspect, wherein the polypeptide of interest is a polypeptide which elicits an immunogenic response in humans. A preferred embodiment of the invention relates to a method of the first aspect, wherein the polypeptide of interest has antimicrobial activity, or wherein the polypeptide of interest is a plant pathogenic polypeptide.

The industrial route to producing the polypeptide of interest in relevant quantities will often involve utilizing recombinant expression systems. Accordingly, a preferred embodiment of the invention relates to a method of the first aspect, wherein an additional step of constructing an expression system is performed, said expression system comprising the polynucleotide isolated in step f).

A second aspect of the invention relates to a polynucleotide encoding a polypeptide of interest, wherein said polynucleotide is isolated by the method of the present invention. A third aspect relates to a polypeptide of interest which is encoded by a polynucleotide as defined in the second aspect.

Nucleic Acid Constructs

The present invention also relates to nucleic acid constructs comprising a nucleic acid sequence of the present invention operably linked to one or more control sequences which direct the expression of the coding sequence in a suitable host cell under conditions compatible with the control sequences. Expression will be understood to include any step involved in the production of the polypeptide including, but not limited to, transcription, post-transcriptional modification, translation, post-translational modification, and secretion. “Expression construct”, “expression vector, or “expression system” are used interchangeably herein, and are defined as a nucleic acid molecule, either single- or double-stranded, which is isolated from a naturally occurring gene or which has been modified to contain segments of nucleic acid combined and juxtaposed in a manner that would not otherwise exist in nature. The term nucleic acid construct is synonymous with the term expression cassette when the nucleic acid construct contains all the control sequences required for expression of a coding sequence of the present invention. The term “coding sequence” is defined herein as a nucleic acid sequence which directly specifies the amino acid sequence of its protein product. The boundaries of the coding sequence are generally determined by a ribosome binding site (prokaryotes) or by the ATG start codon (eukaryotes) located just upstream of the open reading frame at the 5′ end of the mRNA and a transcription terminator sequence located just downstream of the open reading frame at the 3′ end of the mRNA. A coding sequence can include, but is not limited to, DNA, cDNA, and recombinant nucleic acid sequences.

An isolated nucleic acid sequence encoding a polypeptide of the present invention may be manipulated in a variety of ways to provide for expression of the polypeptide. Manipulation of the nucleic acid sequence prior to its insertion into a vector may be desirable or necessary depending on the expression vector. The techniques for modifying nucleic acid sequences utilizing recombinant DNA methods are well known in the art.

The term “control sequences” is defined herein to include all components which are necessary or advantageous for the expression of a polypeptide of the present invention. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide. The term “operably linked” is defined herein as a configuration in which a control sequence is appropriately placed at a position relative to the coding sequence of the DNA sequence such that the control sequence directs the expression of a polypeptide.

The control sequence may be an appropriate promoter sequence, a nucleic acid sequence which is recognized by a host cell for expression of the nucleic acid sequence. The promoter sequence contains transcriptional control sequences which mediate the expression of the polypeptide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al, 1978, Proceedings of the National Academy of Sciences USA 75: 3727-3731), as well as the tac promoter (DeBoer et at, 1983, Proceedings of the National Academy of Sciences USA 80: 21-25). Further promoters are described in “Useful proteins from recombinant bacteria” in Scientific American, 1980, 242: 74-94; and in Sambrook, J. et al., 1989, Molecular Cloning, A Laboratory Manual, 2d edition, Cold Spring Harbor, N.Y.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA which is important for translation by the host cell. The leader sequence is operably linked to the 5′ terminus of the nucleic acid sequence encoding the polypeptide. Any leader, sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 5983-5990.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleic acid sequence encoding the polypeptide would be operably linked with the regulatory sequence.

The present invention also relates to nucleic acid constructs for altering the expression of an endogenous gene encoding a polypeptide of the present invention. The constructs may contain the minimal number of components necessary for altering expression of the endogenous gene. In one embodiment, the nucleic acid constructs preferably contain (a) a targeting sequence, (b) a regulatory sequence, (c) an exon, and (d) a splice-donor site. Upon introduction of the nucleic acid construct into a cell, the construct inserts by homologous recombination into the cellular genome at the endogenous gene site. The targeting sequence directs the integration of elements (a)-(d) into the endogenous gene such that elements (b)-(d) are operably linked to the endogenous gene. In another embodiment, the nucleic acid constructs contain (a) a targeting sequence, (b) a regulatory sequence, (c) an exon, (d) a splice-donor site, (e) an intron, and (f) a splice-acceptor site, wherein the targeting sequence directs the integration of elements (a)-(f) such that elements (b)-(f) are operably linked to the endogenous gene. However, the constructs may contain additional components such as a selectable marker.

The introduction of these components results in production of a new transcription unit in which expression of the endogenous gene is altered. In essence, the new transcription unit is a fusion product of the sequences introduced by the targeting constructs and the endogenous gene. In one embodiment in which the endogenous gene is altered, the gene is activated. In this embodiment, homologous recombination is used to replace, disrupt, or disable the regulatory region normally associated with the endogenous gene of a parent cell through the insertion of a regulatory sequence which causes the gene to be expressed at higher levels than evident in the corresponding parent cell.

The constructs further contain one or more exons of the endogenous gene. An exon is defined as a DNA sequence which is copied into RNA and is present in a mature mRNA molecule such that the exon sequence is in-frame with the coding region of the endogenous gene. The exons can, optionally, contain DNA which encodes one or more amino acids and/or partially encodes an amino acid. Alternatively, the exon contains DNA which corresponds to a 5′ non-encoding region. Where the exogenous exon or exons encode one or more amino acids and/or a portion of an amino acid, the nucleic acid construct is designed such that, upon transcription and splicing, the reading frame is in-frame with the coding region of the endogenous gene so that the appropriate reading frame of the portion of the mRNA derived from the second exon is unchanged.

The splice-donor site of the constructs directs the splicing of one exon to another exon. Typically, the first exon lies 5′ of the second exon, and the splice-donor site overlapping and flanking the first exon on its 3′ side recognizes a splice-acceptor site flanking the second exon on the 5′ side of the second exon, A splice-acceptor site, like a splice-donor site, is a sequence which directs the splicing of one exon to another exon. Acting in conjunction with a splice-donor site, the splicing apparatus uses a splice-acceptor site to effect the removal of an intron.

A fourth aspect relates to an expression system comprising a polynucleotide as defined in the second aspect. A fifth aspect relates to a host cell comprising at least one copy of a polynucleotide as defined in the second aspect, or an expression system as defined in fourth aspect.

Many ways have been described in the art to construct host cells comprising several stable copies of a polynucleotide of interest, both as independently replicating extrachromosomal entities, and as chromosomally stably integrated copies. A preferred embodiment of the fifth aspect relates to the host cell, wherein at least two copies of the polynucleotide as defined in the second aspect are chromosomally integrated.

Process of Production

The present invention also relates to processes for producing a polypeptide of the present invention comprising (a) cultivating a strain, which in its wild-type form is capable of producing the polypeptide, to produce a supernatant comprising the polypeptide; and (b) recovering the polypeptide.

The present invention further relates to methods for producing a polypeptide of the present invention comprising (a) cultivating a homologously recombinant cell, having incorporated therein a new transcription unit comprising a regulatory sequence, an exon, and/or a splice donor site operably linked to a second exon of an endogenous nucleic acid sequence encoding the polypeptide, under conditions conducive for production of the polypeptide; and (b) recovering the polypeptide. The methods are based on the use of gene activation technology, for example, as described in U.S. Pat. No. 5,641,670.

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered by methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedure's (e.g., preparative isoelectric focusing), differential solubility (e.g., ammonium sulfate precipitation), SDS-PAGE, or extraction (see,. e.g., Protein Purification, J. C. Janson and Lars Ryden; editors, VCH Publishers, New York, 1989).

A final aspect relates to a process for producing a polypeptide of interest, comprising cultivating a host cell as defined in the previous aspects under conditions suitable for expressing the polynucleotide of the third aspect, wherein said host cell secretes the polypeptide encoded by said polynucleotide into the growth medium. A preferred embodiment relates to the process of the previous aspect, wherein the polypeptide is an enzyme or a polypeptide having anti-microbial activity. Another preferred embodiment relates to the process of the previous aspect, where an additional step of purifying the polypeptide is performed.

### EXAMPLES

The vector used herein is denoted pMhas5, and the nucleotide sequence is shown in SEQ ID NO:1. The vector has the following features:

| Feature | Location | Description |
|---|---|---|
| CDS | 365-1156 | Kanamycin resistance |
| CDS | 2232-2387 | Beta galactosidase alpha peptide |
| −10 signal | 2189-2192 | Shine Dalgamo |
| Promoter | 2101-2189 | Lac promoter |
| misc feature | 626-650 | KanP1 primer for BACE system |

UV Light

Surprisingly, rolling circle amplification worked on plasmid DNA isolated from E. coli but not on the ligation mixture that was used to transform the E. coli. We determined in a series of experiments, that the reason for why the ligated DNA was not susceptible to rolling circle amplification, was that pyrimidine dimers were present in the ligated DNA. The pyrimidine dimers were formed by exposure of the fragment and plasmid DNA with ultraviolet light before ligation.

A plasmid library was diluted to 1 ng/μl DNA in TE buffer (pH8.0). 10 μl of the diluted plasmid pool was spotted onto a UV source (Eagle Eye™ II UV transilluminator, Stratagene, USA), which was adjusted to preparative mode (UV 360 nm). 2 μl samples were taken every 30 seconds from the irradiated DNA pool, starting with time=0 immediately before turning the UV light on. 10 μl Amersham TempliPhi™ denaturing buffer was then added to each sample and these were then heated to 95° C. for 3 minutes. 10 μl of TempliPhi™ enzyme mix was then added to each sample and the reaction was incubated at 30° C. for 8 hours. The samples were subsequently denatured at 95° C. for 5 minutes, and then stored at 4° C.

Agarose gel loading buffer was added to the samples which were then analyzed in 1% agarose gel electrophoresis according to standard methods. The results showed that the amount of amplification product was significantly reduced in the samples taken after only 30 seconds UV treatment, decreasing until no amplification product was detectable in the sample taken after two minutes of UV irradiation. Presumably modifications of the template DNA had taken place, either by nicking, depurination or pyrimidine dimer formation. The result demonstrated that it is essential to avoid UV treatment of templates to be used in rolling circle amplification.

Library Reparation

5 μg of plasmid pMhas5 was restricted with EcoRI and NotI according to the manufacturers instructions (New England Biolabs, USA). The restricted plasmid was loaded in a standard 1% TBE agarose gel and electrophoresed in order to separate the stuffer fragment from the vector. Half of the gel, containing the DNA marker (1 Kb ladder, BRL, USA), and the region corresponding to approximately 1 mm of the 10 mm total width of the sample lane, was separated using a ruler as a guide. The separated part of the gel was stained with Ethidium bromide and visualized on a UV light source. The band corresponding to the cut vector (ca. 2.6 kb) was marked by making a notch in the gel with a scalpel blade. The two gel parts were aligned and the non UV treated portion corresponding to the cut plasmid was isolated.

A standard GFX™ purification (AP Biotech) was used to remove agarose, restriction enzyme and contaminants. The restricted plasmid was eluted in 50 uls 10 mM Tris buffer pH 8.0 and stored at −20° C. until further use.

A cDNA library was prepared from Rhizomucor pusillus induced for 5 days on Mex-1 media, as shown in the examples of WO 98/38288 and in the examples of international patent application WO 01/12794. The protocols of these PCT publication were used as described, except that care was taken to eliminate all steps involving UV treatment of the RNA, and DNA during the standard procedures. No size fractionation was performed on the RNA, first strand or second strand cDNA. Ligation of the cDNA into pMHas5 was performed according to WO 01/12794. Briefly, 2 μl of vector corresponding to 40 ngs pMHas5 EcoRI-NotI restricted plasmid was used with 6 μl corresponding to about 100 ng of non size-fractionated double-stranded cDNA.

| | | |
|---|---|---|
| pMHas5 | 2 | μl |
| cDNA | 6 | μl |
| 10× lig. Buffer | 1 | μl |
| T4 DNA ligase (3U) | 1 | μl |
| | 10 | μl |

The ligation was incubated at 16° C. overnight and then heat-treated at 65° C. for 20 minutes. 10 μl of dH2O was added to the ligation which was then stored at −20° C. until further use.

Rolling Circle Amplification

1 μl of ligated cDNA library corresponding to about 10 ng total DNA was added to 20 μl TempliPhi™ denaturing buffer (Amersham Biotech). The sample was then heated to 95° C. for 3 minutes and then placed on ice. 10 μl of TempliPhi™ premix was added to the sample which was incubated for 8 hours at 30° C. The sample was then heated to 95° C. for 5 minutes, cooled at 4° C. and stored at −20° C. until further use.

3 μl sample material was restricted with NotI in a total volume of 10 μl under standard conditions (New England Biolabs). 1 μl of the restriction digest mix was visualized on a 1% TBE agarose gel. The gel indicated that amplification had occurred. As expected, a diffuse band between 2.8 and 3.5 kb was observed with tailing into higher molecular weights. This indicated that plasmids with different size inserts were successfully amplified by the method, and that cutting with the unique rarely cutting enzyme NotI created linear monomer plasmids containing the inserts.

The remaining 9 μl of the restriction digest mix was purified on a GFX™ column to remove buffer and enzyme. The sample was eluted from the column with 50 μl buffer (10 mM Tris, pH8.0). 5 μl of the purified sample was analyzed by agarose gel electrophoresis to verify that the amplification product was still present. 20 μl of the purified sample was then used in a standard ligation:

| | | |
|---|---|---|
| GFX purified digest of material: | 20 | μl |
| 10× lig. Buffer | 10 | μl |
| H2O | 69 | μl |
| T4DNA ligase (3U/ul) | 1 | μl |
| | 100 | μl |

The ligated plasmid is expected to contain mainly open circular plasmid monomers with insert. 930 pg of the ligation was used to transform E. coli DH10B electrocompetent cells. 10 pg diluted supercoiled pUCC19 monomer DNA was used as a control. The following results were obtained:

| transformation | transformants/ug DNA |
|---|---|
| ligation | 5.6 × 10⁸ |
| pUC19 | 8.6 × 10⁸ |

This result, clearly demonstrates that a library can be made by in vitro rolling circle amplification. The lower transformation frequency of the treatment results primarily from the fact that the ligation is relaxed plasmid DNA, and not supercoiled as is the pUC19 control. In the literature, the drop in transformation efficiency is reported to be between 100- and 1000-fold, in keeping with what we observed.

Transposon Assisted Signal Trapping and Supercoiling

Transposition with a SigA2 transposon was carried out as described in detail in WO 01/77315, with the following modifications: A period of 2 hours at 30° C. was used for the transposition reaction. Then the sample was split into two portions. The first was treated with a DNA topoisomerase (DNA gyrase) according to the manufacturers instructions (John Innes Enterprizes, England). The second half was left untreated.

It was expected that by supercoiling the plasmid through treatment with a DNA gyrase, one can increase the efficiency of transformation into E. coli by 100-fold. This expectation was confirmed.

Using RecA Recombinase to Create Monomers

An efficient way to create monomers from a rolling circle amplified library is to treat the concatameric DNA with a recombinase enzyme, such as the commercially available RecA. enzyme from E. coli. The result of such a treatment is the looping out of plasmid monomers from the very long linear, amplification products of rolling circle. The method has the advantage that it is not dependent on the use of restriction enzymes that may inadvertently cut the inserted library DNA. The recombinase step may be performed at either simultaneously with the rolling circle amplification, after the amplification, or after transposon treatment.

Reduction of Amplification Bias by Use of Vector Specific Primer Combinations

Although this has not been observed to any large degree, in our experience, selective amplification bias may occur based on differences in the availability of priming sites for the random oligomers used in the Amersham TempliPhi™ premix. In order to reduce this potential bias, several primers specific to the cloning vector may be used. As described in Dean et al. (2001, Rapid Amplification of plasmid and phage DNA using Phi29 DNA, polymerase and multiply-primed rolling circle amplification. Genome Research 11:1095-1099), many primers, priming on both DNA strands are preferable to a one or few primers. In a preferred embodiment, a mixture of 10-20 or more primers are used with an even distribution on the each strand of the cloning vector.

1-44. (canceled)

45. A method for isolating a polynucleotide that encodes a polypeptide of interest which comprises a signal sequence for secretion or partial secretion, the method comprising the sequential steps of:
a) providing a DNA or cDNA library from an organism producing the polypeptide of interest, wherein the library is comprised in a circular vector and is produced in vitro without ultraviolet irradiation of the component polynucleotides;
b) amplifying the library by rolling circle amplification, thereby forming concatamers;
c) inserting into the library a DNA fragment comprising a promoterless and secretion signal-less polynucleotide encoding a secretion reporter;
d) introducing the amplified library comprising the inserted DNA fragment into a host cell;
e) screening for and selecting a host cell that secretes or partially secretes the active secretion reporter; and
f) identifying from the selected host cell the polynucleotide into which the secretion reporter was inserted, and isolating the polynucleotide;
wherein steps b) and c) may be performed in any order.

46. The method of claim 45, wherein the DNA or the cDNA library is normalized.

47. The method of claim 45, wherein the DNA library or cDNA library is derived from a microorganism.

48. The method of claim 47, wherein the microorganism is a fungus, a filamentous fungus or a yeast.

49. The method of claim 47, wherein the microorganism is a bacterium.

50. The method of claim 47, wherein the microorganism is an archaeon.

51. The method of claim 45, wherein the DNA library or cDNA library is derived from a multicellular organism.

52. The method of claim 45, wherein the vector comprises at least one restriction enzyme cleavage site and/or at least one cos site and/or at least one recombination recognition site.

53. The method of claim 45, wherein step c) is performed in vitro.

54. The method of claim 45, wherein the DNA fragment comprises a transposon.

55. The method of claim 45, wherein the DNA fragment comprises an origin of replication which is functional in the host cell.

56. The method of claim 45, wherein the secretion reporter is a protein which, when secreted from the host cell, allows said cell to grow in the presence of a substance which otherwise inhibits growth of said cell.

57. The method of claim 56, wherein the secretion reporter is a □-lactamase or an invertase.

58. The method of claim 45, wherein the polynucleotide of the DNA-fragment of steb (b) encodes a secretion reporter carrying an N-terminal peptide linker which comprises a specific target site for proteolytic cleavage.

59. The method of claim 45, wherein the amplified library concatamers are converted to monomers before performing step d).

60. The method of claim 45 wherein the vector comprises at least one restriction enzyme recognition site, and the concatamers are converted to monomers by restriction enzyme digestion and then circularized by ligation.

61. A polynucleotide encoding a polypeptide of interest, wherein said polynucleotide is isolated by the method of the present invention.

62. A polypeptide of interest which is encoded by the polynucleotide of claim 61.

63. A host cell comprising at least one copy of the polynucleotide of claim 61.

64. A process for producing a polypeptide of interest, comprising cultivating the host cell of claim 64 under conditions suitable for expressing the polynucleotide, wherein said host cell secretes the polypeptide encoded by said polynucleotide into the growth medium.
